(19) United States
(12) Patent Application Publication
(10) Pub. No.: US 2021/0147773 A1
(43) Pub. Date: May 20, 2021

(54) DEVICE FOR SUPPORTING DEVELOPMENT OF A CELL OR TISSUE DERIVED FROM THE OVARY

(71) Applicant: University of Leeds, Leeds, Yorkshire (GB)

(72) Inventors: Virginia PENSABENE, Leeds, Yorkshire (GB); Helen Mary PICTON, Leeds, Yorkshire (GB); Paul Joseph MCKEEGAN, Leeds, Yorkshire (GB); Francesco COLUCCI, Leeds, Yorkshire (GB)

(73) Assignee: University of Leeds, Leeds, Yorkshire (GB)

(21) Appl. No.: 17/046,834

(22) PCT Filed: Apr. 18, 2019
(86) PCT No.: PCT/GB2019/051113

§ 371 Date: Oct. 12, 2020

(30) Foreign Application Priority Data
Apr. 18, 2018 (GB) 1806309.9
Apr. 18, 2018 (GB) 1806311.5

(51) Int. Cl. C12M 3/00; C12M 3/06

(52) U.S. Cl. CPC C12M 21/06; C12M 23/16

(57) ABSTRACT

A device for supporting development of a cellular deposit comprising at least one of a cell or a tissue derived from an ovary. The device comprises an inlet well, an outlet well, and an enclosed culture chamber disposed between the inlet well and the outlet well. The device further comprises an inlet channel fluidly coupling the inlet well to the culture chamber and an outlet chamber and/or at least one outlet channel fluidly coupling the culture chamber to the outlet well. At least one of the outlet chamber and the at least one outlet channel is sized to prevent passage of the cellular deposit therethrough.

The present invention relates to a device for supporting development of at least one of a cell or tissue derived from the ovary. In particular, but not exclusively, embodiments of the present invention relate to a microfluidic device for supporting embryo development. A system comprising the device and corresponding uses of the device are also provided.

The dynamic processes by which a fertilized egg becomes an embryo, called embryogenesis, is characterized by a series of precisely timed and coordinated events that will determine which embryos have the capacity to undergo genome activation, develop to the blastocyst stage and later implant to produce a pregnancy and yield healthy offspring.

Prior to fertilization, the egg (oocyte) divides by meiosis, enlarges, and matures in the ovarian follicle until it reaches a stage of meiotic division called metaphase II. At this point, the oocyte is released into the oviduct, the tube-like structure that connects the ovary to the uterus, and is fertilized. After fertilization, the mammalian zygote starts its journey to the uterus, a journey that takes from 3 to 7-8 days in, for example, species such as mouse, cow and human, during which preimplantation embryos can experience fluid flow and sense shear stress as a consequence of propulsion by cilia and smooth muscle-mediated peristalsis. As it travels, the zygote divides. The first cleavage produces two identical cells and then divides again to produce 4 cells, 8 cells, 16 cells (morula stage), and so on until the blastocyst stage is reached. For example, in species such as mice, cow and human, the division of the outer cells of the 16-cell morula produces an outer rim of cells (the trophectoderm) and an inner core of cells (the inner cell mass) which is the first evidence of cell specialization. Parallel to the physical separation and differentiation of the trophectoderm from the inner cell mass, the fluid-filled blastocoel cavity forms and the morula becomes a blastocyst. For example, in mice, cow and human, at the blastocyst stage (or shortly after) the embryo reaches the uterus.

8-cell stage embryos can be more sensitive to shear stress than the early blastocyst stage. Chronic shear stress (1.2 dyne cm-2 within 12 h) can be lethal at the blastocyst stage, and the proteinaceous zona pellucida coat deadens shear stress effects. When it arrives in the uterus, the blastocyst “hatches” out of the zona pellucida, the structure that originally surrounds the oocyte and that also prevents the implantation of the blastocyst into the wall of the oviduct.

The emerging field of microfluidics, makes it possible to successfully reproduce the physiological conditions of preimplantation development in vitro. While microfluidics was initially developed in the mid-1970s, the first “lab on chip” devices in the early 1990s, able to reduce conventional laboratory equipment into miniaturized platforms integrating microfluidic design with biology, especially for research in cell biology. Microfluidic systems could support several parts of Assisted Reproductive Technology (ART) treatments in a closed and optimised system. Gamete selection, fertilization and preimplantation development have all been successfully performed in a microfluidic system. Thanks to computational model analysis, microfluidic devices offer the possibility to customise and optimise the microfluidic design before fabrication, reducing in this way handling, stress and human error associated with traditional culture techniques (e.g. microdrops under oil). Current methods for in vitro embryo culture are mainly based on embryos in petri dishes in drops of 20 to 100 μL of defined culture medium, covered by mineral or paraffin oil to avoid evaporation.

Mechanical stress (induced by moving embryos using embryo handling pipettes) or changes in the culture environment (e.g. media, pH, temperature, O₂, tension, additives) can have a significant, negative effect on the health of the embryos. Within a microfluidic device, the ability to assess and manipulate embryos/cells and their microenvironment, both mechanically and biochemically, could provide a greater understanding of how to optimise ART, and so help the development of a more physiologically relevant embryo culture system.

Several microfluidic devices have been developed with embryos cultured in microchannels or microchambers, secured in place through use of a well-like structure or valves (see, for example, US2007/0231901 A1, U.S. Pat. No. 6,695,765 B1 and WO2011/160430 A1).

According to a first aspect of the present invention, there is provided a device for supporting development of a cellular deposit comprising at least one of a cell or a tissue derived from an ovary, the device comprising:
an inlet well, an outlet well, and an enclosed culture chamber disposed between the inlet well and the outlet well;
an inlet channel fluidly coupling the inlet well to the culture chamber;
an outlet vessel (or receptacle) fluidly coupling the culture chamber to the outlet well;

wherein the outlet vessel (or receptacle) is sized to prevent passage of the cellular deposit therethrough.

Aptly, the outlet vessel is an outlet chamber and/or at least one outlet channel. In other words, there is provided a device for supporting development of a cellular deposit comprising at least one of a cell or a tissue derived from an ovary, the device comprising:
an inlet well, an outlet well, and an enclosed culture chamber disposed between the inlet well and the outlet well;
an inlet channel fluidly coupling the inlet well to the culture chamber;
an outlet chamber and/or at least one outlet channel fluidly coupling the culture chamber to the outlet well;

wherein at least one of the outlet chamber and the at least one outlet channel (i.e. each of the at least one outlet channels) is sized to prevent passage of the cellular deposit therethrough.

That is, the device may comprise at least one outlet channel fluidly coupling the culture chamber to the outlet well, wherein each of the at least one outlet channel is sized to prevent passage of the cellular deposit therethrough.

Alternatively, the device may comprise an outlet chamber fluidly coupling the culture chamber to the outlet well (for example via at least one outlet channel), wherein the outlet chamber (and also optionally at least one outlet channel) is sized to prevent passage of the cellular deposit therethrough. Suitably such a device may also include a plurality of inlet wells, a plurality of culture chambers, each culture chamber being disposed between a corresponding inlet well and the outlet well, and a plurality of inlet channels, each inlet channel fluidly coupling a corresponding inlet well to its corresponding culture chamber. The outlet chamber may fluidly couple each culture chamber to the outlet well.

Suitably, the device is microfluidic.

Suitably, the inlet channel is configured for flow of fluid by capillary action between the inlet well and the culture chamber.

Suitably, each of the at least one outlet channel is configured for flow of fluid by capillary action between the culture chamber and the outlet well.

Suitably, the inlet channel extends from a lower portion of the inlet well to an upper portion of the culture chamber.

Suitably, a lower wall of the inlet well is coplanar with a lower wall of the inlet channel.

Suitably, an upper wall of the inlet channel is coplanar with an upper wall of the culture chamber.

Suitably, each of the at least one outlet channel extends from a lower portion of the culture chamber to a lower portion of the outlet well.

Suitably, a lower wall of the culture chamber is coplanar with a lower wall of each of the at least one outlet channel.

Suitably, the lower wall of each of the at least one outlet channel is coplanar with a lower wall of the outlet well.

Suitably, a height and/or a width of each of the at least one outlet channel is less than a diameter of the cellular deposit.

Suitably, the height and/or the width of each of the at least one outlet channel is less than or equal to 50% of the diameter of the cellular deposit.

Suitably, the inlet channel is sized to allow passage of a developed material.

Suitably, a smallest cross-sectional dimension of the inlet channel is at least 1.5 times larger than the diameter of the developed material.

Suitably, the culture chamber is substantially circular, oval or elliptical in shape and has a constant height.

Suitably, a diameter or width of the culture chamber is greater than or equal to a width of the inlet channel.

Suitably, the culture chamber has a volume from about 3nL to about 100 μL.

Suitably, the inlet well and the outlet well each have a volume from about 2 to about 20 μL.

Suitably, the inlet well comprises an inclined lower portion, wherein the inclined lower portion is inclined towards the opening of the inlet channel at an angle between 0 and 90 degrees.

Suitably, the inlet channel has a volume substantially equal to a total volume of the at least one outlet channel.

Suitably, the device comprises a plurality of outlet channels.

Suitably, the plurality of outlet channels each extend substantially in parallel between the culture chamber and the outlet well.

Suitable, at least one of the plurality of outlet channels has a different volume compared to another one of the plurality of outlet channels.

Suitably, the device further comprises a reservoir defined between an outer wall of the device and an inner wall spaced apart from the outer wall, wherein the reservoir extends around the inlet well, outlet well, culture chamber, inlet channel and at least one outlet channel and is configured to retain a volume of fluid therein.

According to a second aspect of the present invention there is provided use of the device of any preceding claim for supporting development of a cellular deposit comprising at least one of a cell or a tissue derived from an ovary.

Suitably, the cell or the tissue derived from the ovary is selected from an oocyte, an ovarian follicle, ovarian tissue and an embryo, optionally wherein the embryo is selected from a zygote, zona pellucida, a cleavage stage embryo, a morula, and a blastocyst.

According to a third aspect of the present invention there is provided a microfluidic device for supporting embryo development, the device comprising:
an inlet well, an outlet well, and an enclosed culture chamber disposed between the inlet well and the outlet well;
an inlet channel fluidly coupling the inlet well to the culture chamber; at least one outlet channel fluidly coupling the culture chamber to the outlet well;
wherein each of the at least one outlet channel is sized to prevent passage of a zygote stage embryo therethrough.

Suitably, the inlet channel is configured for flow of fluid by capillary action between the inlet well and the culture chamber.

Suitably, each of the at least one outlet channel is configured for flow of fluid by capillary action between the culture chamber and the outlet well.

Suitably, the inlet channel extends from a lower portion of the inlet well to an upper portion of the culture chamber.

Suitably, a lower wall of the inlet well is coplanar with a lower wall of the inlet channel.

Suitably, an upper wall of the inlet channel is coplanar with an upper wall of the culture chamber.

Suitably, each of the at least one outlet channel extends from a lower portion of the culture chamber to a lower portion of the outlet well.

Suitably, a lower wall of the culture chamber is coplanar with a lower wall of each of the at least one outlet channel.

Suitably, the lower wall of each of the at least one outlet channel is coplanar with a lower wall of the outlet well.

Suitably, a height and/or a width of each of the at least one outlet channel is less than a diameter of the zygote stage embryo.

Suitably, the height and/or the width of each of the at least one outlet channel is less than or equal to 50% of the diameter of the zygote stage embryo.

Suitably, the inlet channel is sized to allow passage of a blastocyst stage embryo.

Suitably, a smallest cross-sectional dimension of the inlet channel is at least 1.5 times larger than the diameter of a blastocyst stage embryo.

Suitably, the culture chamber is substantially circular, oval or elliptical in shape and has a constant height.

Suitably, a diameter or width of the culture chamber is greater than or equal to a width of the inlet channel.

Suitably, the culture chamber has a volume from about 3 nL to about 100 μL.

Suitably, the inlet well and the outlet well each have a volume from about 2 to about 20 μL.

Suitably, the inlet well comprises an inclined lower portion, wherein the inclined lower portion is inclined towards the opening of the inlet channel at an angle between 0 and 90 degrees.

Suitably, the inlet channel has a volume substantially equal to a total volume of the at least one outlet channel.

Suitably, the device comprises a plurality of outlet channels.

Suitably, wherein the plurality of outlet channels each extend substantially in parallel between the culture chamber and the outlet well.

Suitably, at least one of the plurality of outlet channels has a different volume compared to another one of the plurality of outlet channels.

Suitably, the device further comprises a reservoir defined between an outer wall of the device and an inner wall spaced apart from the outer wall, wherein the reservoir extends around the inlet well, outlet well, culture chamber, inlet channel and at least one outlet channel and is configured to retain a volume of fluid therein.

According to a fourth aspect of the present invention there is provided use of the device of the first aspect for culturing at least one embryo.

Suitably, at least one embryo is a mammalian embryo, optionally wherein at least one embryo is selected from a human or other primate, rodent, cattle, sheep or pig embryo.

Certain embodiments of the present invention provide a device that helps to regulate and better control the embryo microenvironment.

Certain embodiments of the present invention provide a device for culture of an embryo that can be used without the need for a mineral oil layer, which can be deleterious to the health of the embryo.

Certain embodiments of the present invention help to mimic the natural fluid flow that draws embryos through the fallopian tube to the site of implantation in the uterus.

Certain embodiments of the present invention provide a device that enables transport of embryos whilst minimising mechanical stresses on the embryos (for example, reduction in shear stress, mechanical strain etc.)

Certain embodiments of the present invention allow for a reduction of media evaporation from the device compared to conventional devices.

Certain embodiments of the present invention allow reduced handling of medium containing embryos during the preimplantation embryo development phase in vitro.

Certain embodiments of the present invention allow for a reduction in the media volume used during the culture process. For example, the volume of media used may be around 0.01 to 10 μL compared to 20 to 100 μL used in many conventional devices.

Embodiments of the invention are further described hereinafter with reference to the accompanying drawings, in which:

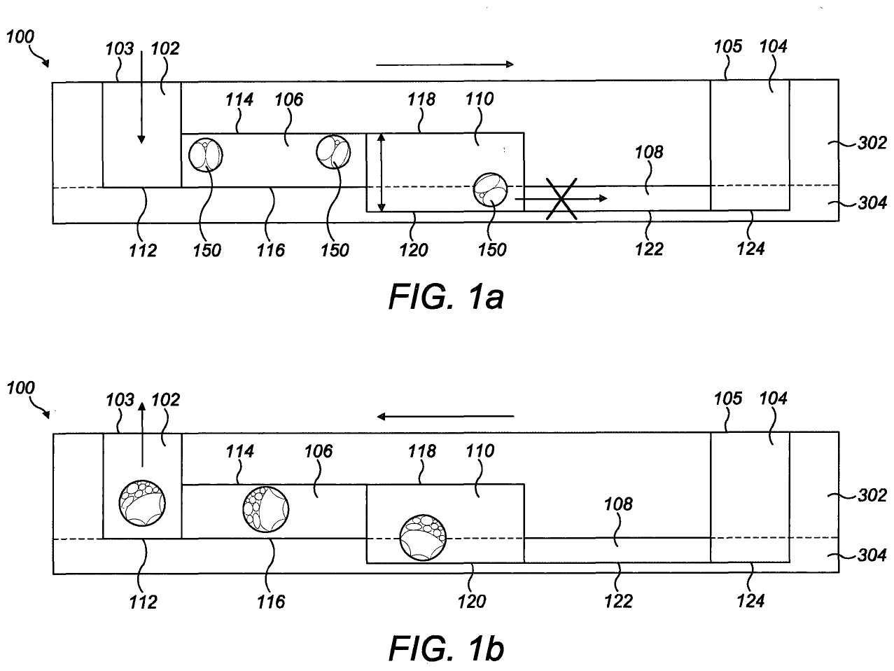

FIG. 1a illustrates a microfluidic device and embryo loading into the microfluidic device;

FIG. 1b illustrates embryo retrieval from the microfluidic device of FIG. 1a;

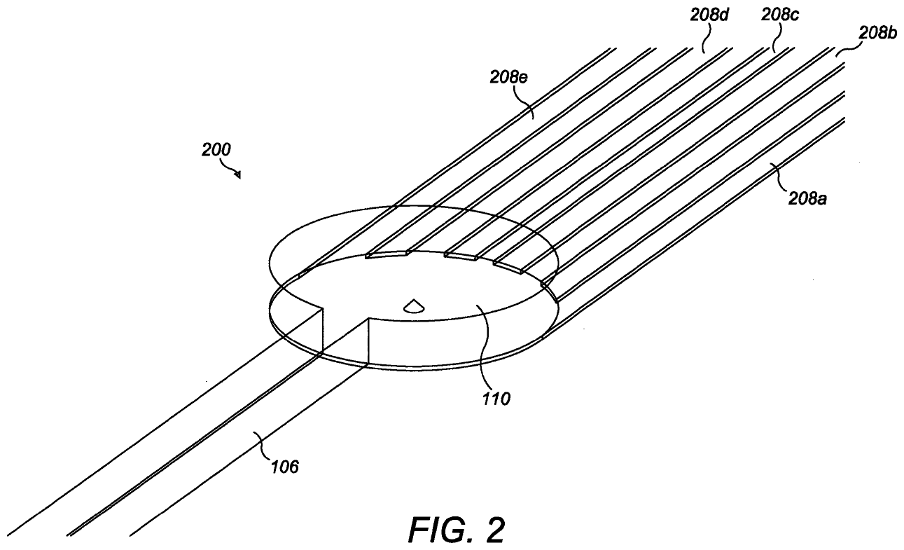

FIG. 2 illustrates a portion of an alternative microfluidic device;

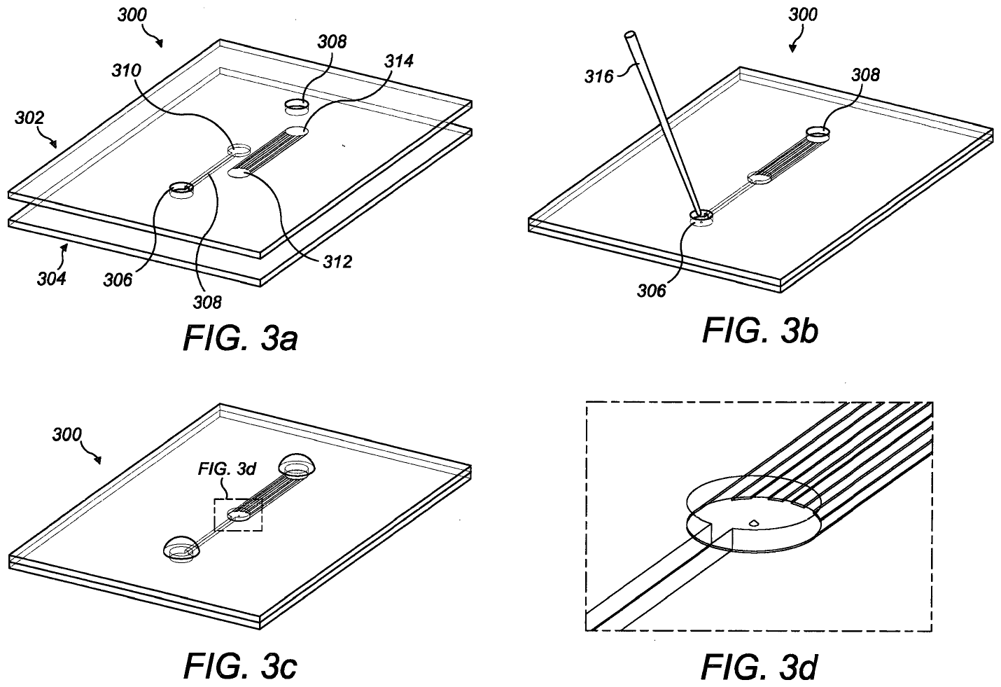

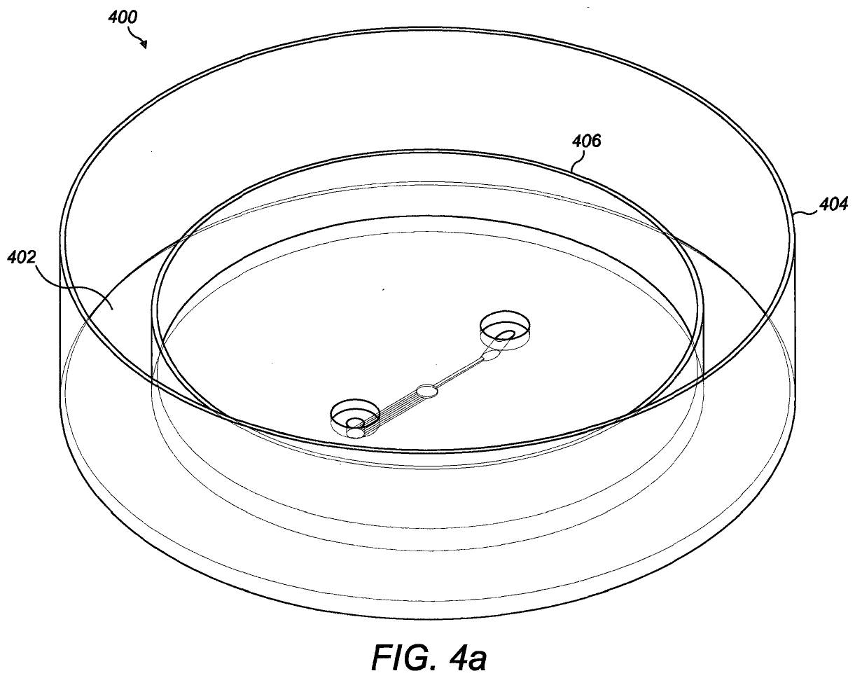

FIGS. 3a to 3d illustrate another example of a microfluidic device;

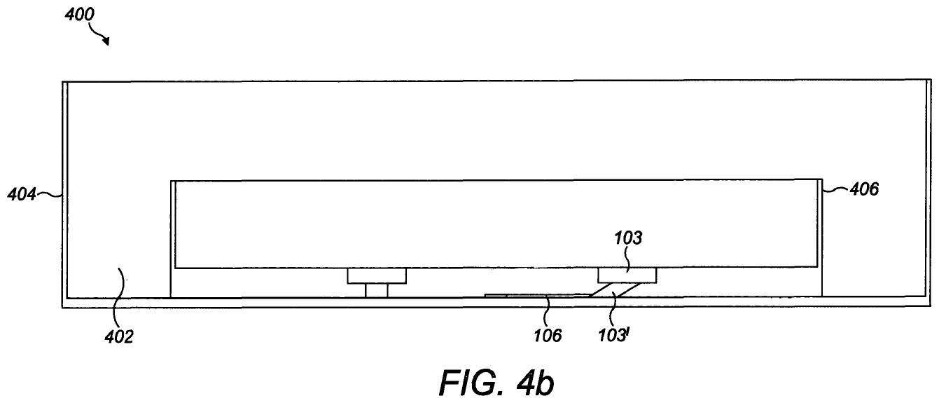

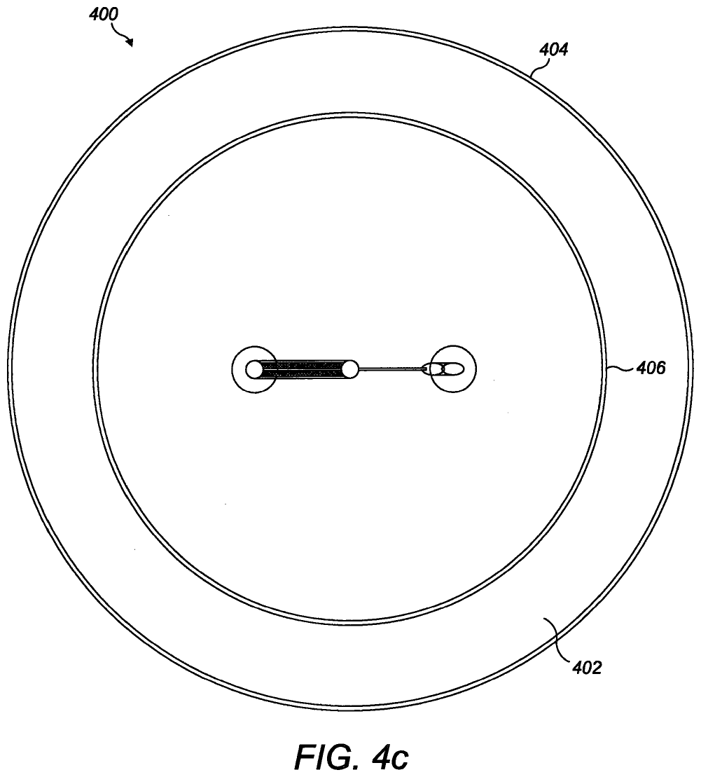

FIGS. 4a to 4c illustrate a system including a microfluidic device in perspective, side and plan views respectively;

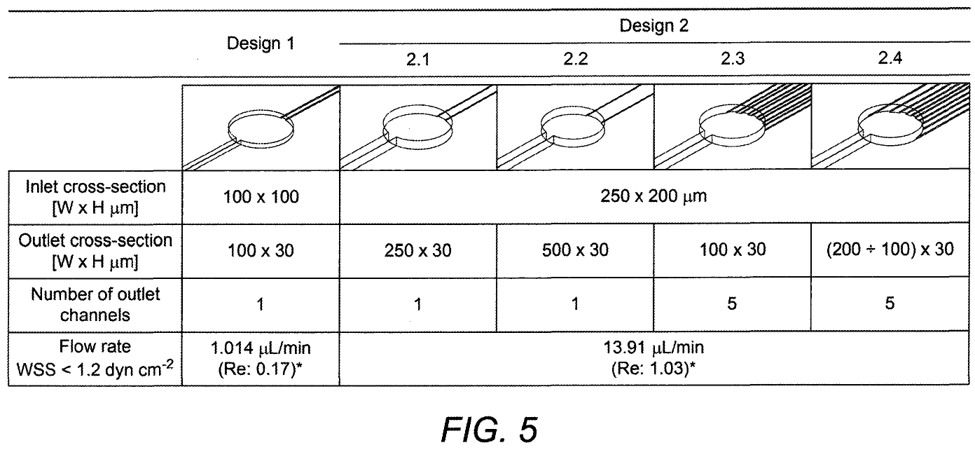

FIG. 5 is a table illustrating the flow rate of different designs of microfluidic devices;

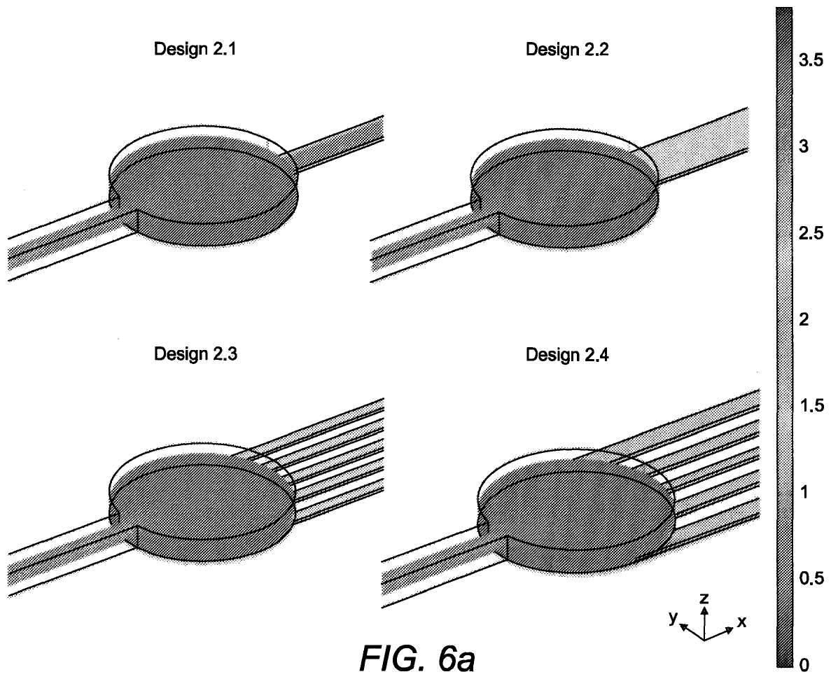

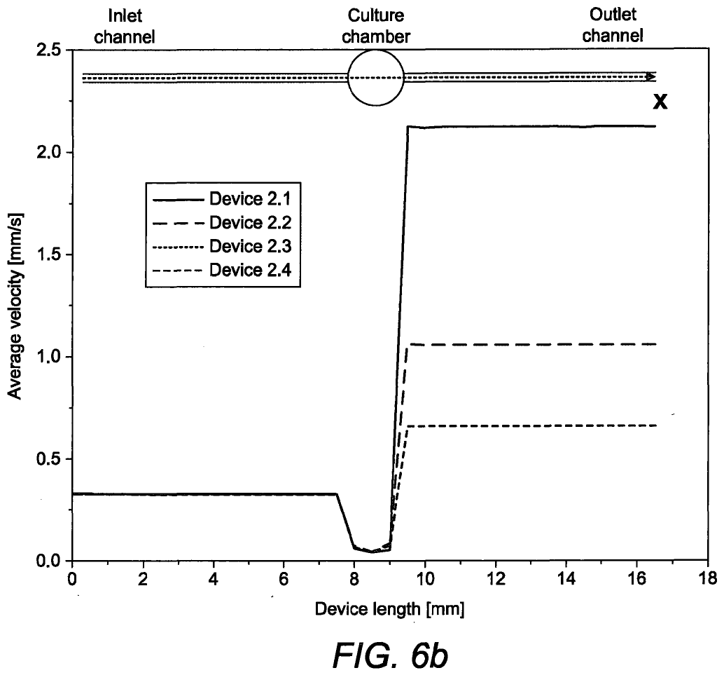

FIG. 6a is a fluid flow analysis of the velocity generated in the microfluidic devices with an inlet flow rate of 1.014 μL min⁻¹ (τ=1.2 dyn cm⁻²), with the colour spectrum bar showing the velocity in mm/s;

FIG. 6b is a plot of the average velocity across the device length x, from inlet to outlet channel;

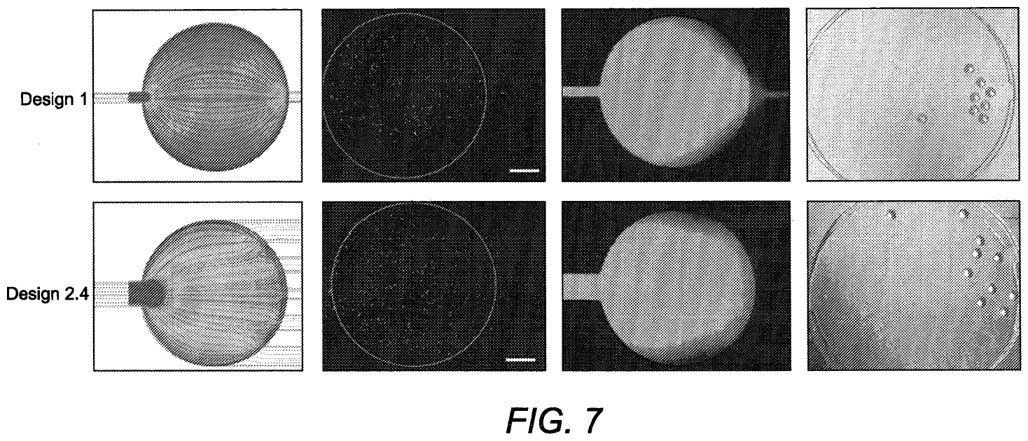

FIG. 7 shows flow, gradients and beads distribution in the microfluidic chamber in the Design 1 (top row) and in Design 2.4 (low row), scale bar is 200 μm;

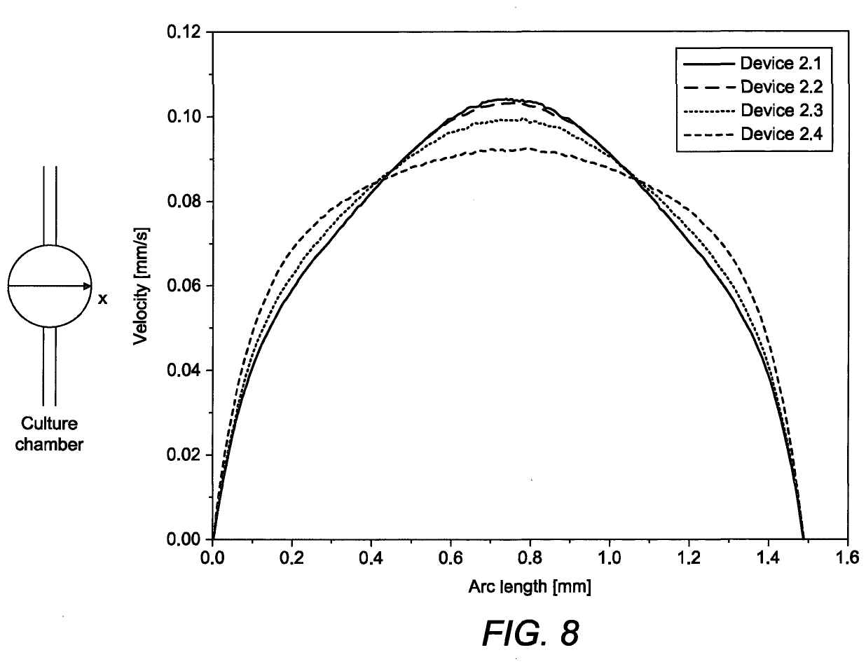

FIG. 8 shows computationally estimated velocity profiles across the length of the middle culture chamber, where arc length refers to the chamber width at the middle height as represented by the arrow;

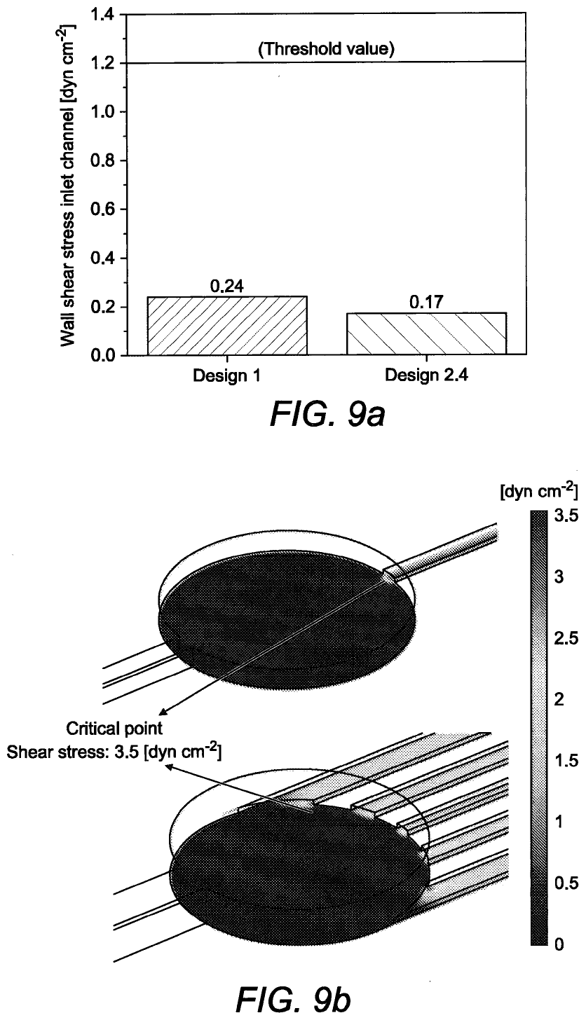

FIG. 9a is a computationally estimated inlet channels wall shear stress for design 1 and design 2.4;

FIG. 9b is a fluid flow computational model of shear stress field surface plot during manual loading, wherein the colour spectrum bar shows the shear stress field generated in the fluid systems;

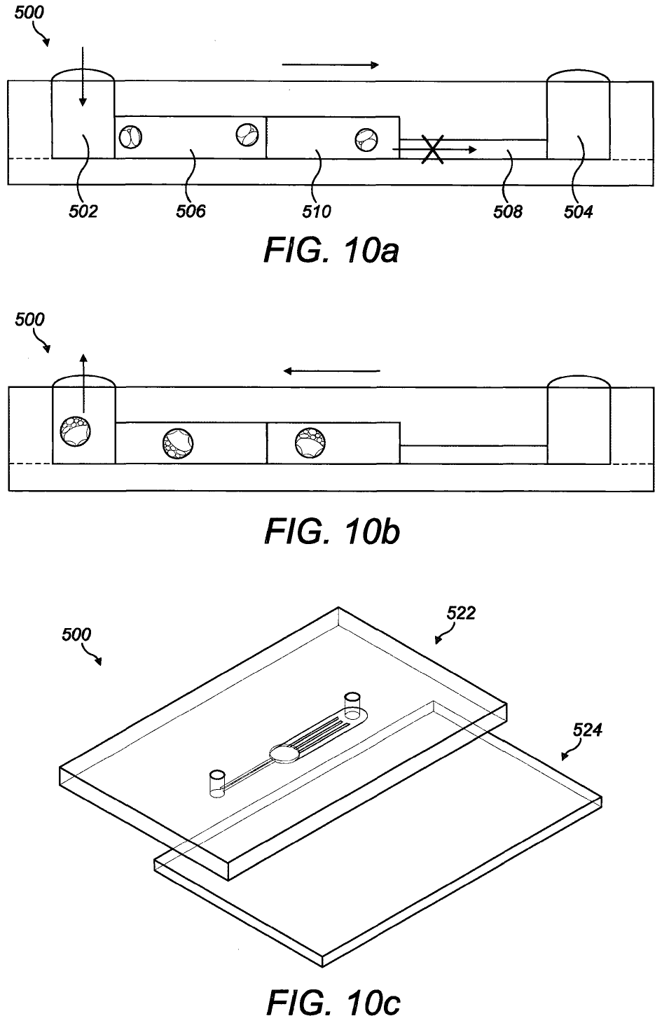

FIGS. 10a to 10c illustrate another example of a microfluidic device; and

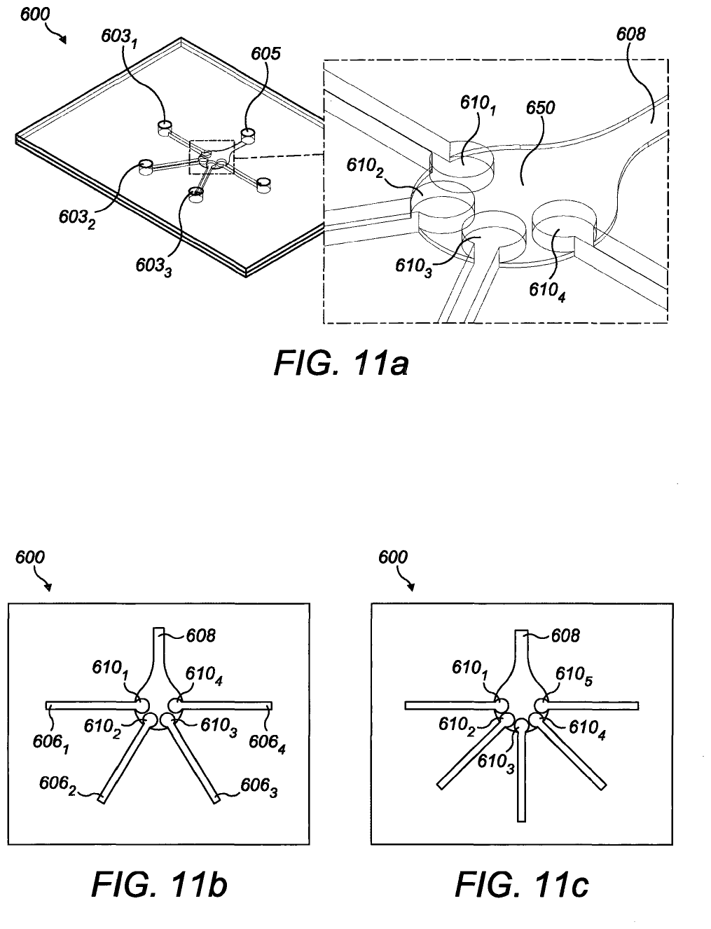

FIGS. 11a to 11c illustrate another example of a microfluidic device.

In the drawings like reference numerals refer to like parts.

The devices described herein may be used to support the development of any cells and/or tissues, for example, cells and/or tissues that are derived from an ovary.

As used herein, the term “derived from an ovary” encompasses any cell and/or tissue that directly or indirectly originates from the ovary, including differentiated products thereof. For example, oocytes, ovarian follicles (such as primordial follicles, or cumulus-oocyte complexes, preantral follicles or antral follicles) and ovarian tissue (e.g. ovarian tissue fragments) are all encompassed by this term. In addition, embryos are encompassed, as they are originally derived from an ovarian product (the oocyte) and thus are indirectly derived from the ovary. As used herein, the term “embryo” encompasses zygote, zona pellucida, cleavage stage embryo, morula, and blastocyst.

As used herein, the term “supporting the development” refers to maintaining the viability of the cell and/or tissue that is deposited in the device and supporting cell growth, cell division and/or cell differentiation of the cellular material. Cells and/or tissue that are deposited in the device are also referred to herein as the “cellular deposit” (i.e. the cellular material that is deposited into the device, prior to any cell division and/or cell differentiation within the device). Typically, the cellular deposit is a multicellular deposit (e.g. a multi-cellular product derived from the ovary).

Accordingly, the device is suitable for supporting the development of a cellular deposit (e.g. zygote stage embryo) comprising at least one of a cell or a tissue derived from an ovary.

Typically, the cellular deposit develops within the device (by cell growth and cell division and/or cell differentiation) to form a developed material. As used herein, the term “developed material” refers to the cells/and or tissue that are to be retrieved from the device (e.g. blastocyst stage embryo).

The following examples are described with reference to development of an embryo. However, it will be appreciated that the device may be adapted for culture of other cells or tissue types. For example, the device may be made larger or smaller to accommodate tissues or cells of different sizes. Alternative devices suitable for other tissue and/or cell types are discussed under “Alternative devices” below.

FIGS. 1a and 1b illustrate an example of a microfluidic device 100 for supporting embryo development. As used herein, the term “microfluidic device” refers to a device having at least one flow channel or conduit with a single dimension of less than 1000 micrometres, wherein the single dimension referred to is the height or width of the conduit, and not its length.

The microfluidic device 100 includes an inlet well 102, an outlet well 104 and a culture chamber 110 disposed between the inlet well 102 and the outlet well 104. The microfluidic device 100 also includes an inlet channel 106 that fluidly couples the inlet well 102 to the culture chamber 110, and an outlet channel 108 that fluidly couples the culture chamber 110 to the outlet well 104. The culture chamber 110 is enclosed with the inlet channel 106 and the outlet channel 108 providing fluid flow paths for fluid from the inlet channel and/or the outlet channel to enter and/or exit the culture chamber 110.

The inlet well 102 and the outlet well 104 are each configured to hold a predetermined volume of fluid. Each of the inlet well 102 and the outlet well 104 include an open top 103, 105 into which fluid may be added and/or removed from the well (e.g. using a suitably sized pipette). Fluid may also enter and/or exit the inlet well 102 via the inlet channel 106 that is fluidly coupled to the inlet well 102. Similarly, fluid may also enter and/or exit the outlet well 104 via the outlet channel 108 that is fluidly coupled to the outlet well 104.

The outlet channel 108 is sized to prevent passage of a zygote stage embryo (i.e. the cellular deposit) therethrough. In this way, as illustrated in FIG. 1a, a zygote stage embryo 150 can be loaded into the device 100 at the open top 103 of the inlet well 102 (as indicated by the arrow). The embryo 150 continues along the inlet channel 106 to the culture chamber 110. The embryo 150 cannot exit the culture chamber 110 via the outlet channel 108 since the outlet channel 108 is sized smaller than the embryo 150. The outlet channel 108 blocks the passage of the embryo 150 (i.e. the cellular deposit) therethrough, thereby acting to retain the embryo 150 in the culture chamber 110.

In this example, the height (i.e. thickness) of the outlet channel 108 is less than a diameter of the zygote stage embryo 150. In other examples, the width or the height and the width of the outlet channel 108 may each be less than the diameter of the zygote stage embryo 150 (i.e. the cellular deposit). It will be appreciated that embryos of different species will have different diameters at the zygote stage. As such, the height and/or the width of the outlet channel 108 may be selected according to the species of embryo to be used in the device. For example, a mouse embryo has a diameter of around 60 μm at the zygote stage. As such, in a device for use with a mouse embryo, the outlet channel 108 will be configured to have a height and/or width of less than 60 μm.

Aptly, the height and/or width of the outlet channel 108 may be less than or equal to 50% of the diameter of the zygote stage embryo 150. For example, in a device for use with a mouse embryo, the outlet channel 108 may be configured with a height and/or width of less than or equal to 30 μm. It will be appreciated that the height and/or width of the outlet channel may be scaled according to the embryo species to be used with the device (or different cellular deposits to be used within the device).

The inlet channel 106 is aptly configured for flow of fluid by capillary action between the inlet well 102 and the culture chamber 110. As such, fluid (optionally containing one or more embryos or other cellular deposit) may be deposited within the inlet well 102 via the open top 103. The fluid will then be drawn along the inlet channel 106 by capillary action towards the culture chamber 110. This process can also work in reverse by removing fluid from the inlet well 102 via the open top 103 such that fluid in the culture chamber 110 is drawn along the inlet channel 106 by capillary action towards the inlet well 102.

In this example, the outlet channel 108 is also configured for flow of fluid by capillary action between the culture chamber 110 and the outlet well 104. As such, fluid in the culture chamber 110 can be drawn along the outlet channel 108 by capillary action towards the outlet well 104. This process can also work in reverse by removing fluid from the inlet well 102 such that fluid in the culture chamber 110 is drawn along the inlet channel 106 by capillary action towards the inlet well 102, thereby removing fluid from the culture chamber 110, which will consequently draw fluid from the outlet well 104 by capillary action towards the culture chamber 110. Of course, in another example, the culture chamber 110 may initially be empty of fluid (or substantially empty) and fluid may be deposited in the outlet well 104 at the open top 105. The fluid deposited in the outlet well 104 may then be drawn towards the culture chamber 110 by capillary action through the outlet channel 108.

As such, it will be appreciated that flow of fluid through the device can be controlled by adjusting the levels of fluid in each of the inlet well 102 and the outlet well 104 by removing and/or depositing fluid via the open tops 103, 105.

In this example, the inlet channel 106 extends from a lower portion of the inlet well 102. That is, the inlet channel 106 is fluidly coupled to the inlet well 102 at a lower end of the inlet well 102. As such, the inlet channel 106 is configured to draw fluid from the bottom of the inlet well 102. The inlet channel 106 extends from the lower portion of the inlet well 102 to an upper portion of the culture chamber 110. As such, fluid exits the inlet channel 106 at an upper portion of the culture chamber 110. Any embryos (i.e. cellular deposits) present in the fluid will tend to settle in the lower portion (i.e. the bottom) of the culture chamber 110. As such, in the absence of fluid flow in a direction towards the inlet well 102, the embryos 150 are retained in the culture chamber 110.

A lower wall 112 (i.e. the bottom) of the inlet well 102 is aptly coplanar (i.e. continuous with) a lower wall 116 of the inlet channel 106. An upper wall 114 (i.e. top) of the inlet channel 106 is aptly coplanar (i.e. continuous with) an upper wall 118 of the culture chamber 110. This may help to optimise fluid flow and passage of embryos 150 through the device 100 into the culture chamber 110.

The outlet channel 108 aptly extends from a lower portion of the culture chamber 110 to a lower portion of the outlet well 104. Aptly, a lower wall 120 (i.e. bottom) of the culture chamber 110 is coplanar (i.e. continuous with) a lower wall 122 of the outlet channel 122 and a lower wall 124 of the outlet well 104. This can help to aid flow of fluid from the culture chamber 110 to the outlet well 104 by capillary action.

In this example, the culture chamber 110 is substantially circular in shape and has a constant height h. The height h of the culture chamber 110 is aptly equal to the height of the inlet channel 106 plus the height of the outlet channel 108. In other examples, the culture chamber may have a different shape (e.g. oval, elliptical, rectangular, or other polygonal shape). It will be appreciated that the culture chamber may be sized to accommodate the embryo (or other cellular deposit) to be deposited in the device and also to accommodate the embryo (or other developed material) to be retrieved from the device.

Aptly, a diameter or width of the culture chamber is greater than or equal to a width of the inlet channel.

The culture chamber 110 may have a volume from about 3 nL to about 100 μL. In a device designed for use with a mouse embryo, the volume of the culture chamber may be around 400 to 410 nL, for example.

Each of the inlet well 102 and the outlet well 104 may have a volume larger than the volume of the culture chamber 110.

The volume of the inlet channel 106 and the volume of the outlet channel 108 is aptly less than the volume of the culture chamber 110. For example, the volume of the inlet channel 106 and the volume of the outlet channel 108 may be less than 70% of the volume of the culture chamber 110. For example, the inlet channel and the outlet channel may each have a volume from around 200 to 500 nL. In a device designed for use with a mouse embryo, the volume of each of the inlet and outlet channels may be from around 350 to 400 nL, for example. Aptly, the volume of the inlet channel is equal to the volume of the outlet channel.

In using the device 100, an embryo culture development media fluid (e.g. KSOM) is added to the inlet well 102. This fluid is drawn through the device 100 several times (by loading fluid into the inlet well 102 via the open top 103 and removing the outlet well 104 via the open top 105 accordingly), until the culture chamber 110, the inlet channel 106 and the outlet channel 108 are each full of the fluid. To load one or more embryos 150 (i.e. the cellular deposit) into the device 100, the embryo(s) are deposited in the inlet well 102. Fluid is removed from the outlet well 104 via the open top 105 to start flow of fluid through the device 100 in a direction from the inlet well 102 to the outlet well 104 (as indicated by the arrows in FIG. 1a). As such, the fluid in the inlet well 102 (including the embryo(s) or other cellular deposit) is drawn through the inlet channel 106 via capillary action into the culture chamber 110. This flow process is continued until all the embryo(s) have entered the culture chamber 110. Extra fluid is then deposited in the inlet well 102 and/or the outlet well 104 before culture of the embryos 150.

The embryo(s) may typically be retrieved from the device at the blastocyst stage (i.e. the developed material). Though in some examples, retrieval of the embryos at other development stages may be applicable. As such, the inlet channel 106 is aptly sized to allow passage of a blastocyst stage embryo (i.e. the developed material) therethrough. It will be appreciated that embryos of different species will have different sizes at the blastocyst stage, and as such, the inlet channel 106 may be sized appropriately according to the embryo species to be cultured in the device. In other examples, the embryo may be retrieved from the device at a later or earlier stage of development (e.g. at the morula or the hatched blastocyst stage). In this example, it will be appreciated that the inlet channel may be sized accordingly to allow passage of the smaller or larger embryo upon retrieval.

Aptly, the inlet channel 106 may be sized such that the smallest cross-sectional diameter (e.g. the height and/or width) is at least 1.5 times larger than the diameter of a blastocyst stage embryo (or other developed material). This may help to allow easy passage of the blastocyst embryo (or other developed material) through the inlet channel, without imparting excessive shear stress or other mechanical stresses on the embryo, for example.

To retrieve the embryos (or other developed material), fluid is aspirated (i.e. removed from) the inlet well 102 via the open top 103 using, e.g. a pipette (see FIG. 1b). Fluid then begins to flow in a direction from the outlet well 104 to the inlet well 106 through the outlet channel 108, the culture chamber 110 and the inlet channel 106, thereby transporting the embryos (or other developed material) towards the inlet well 102. The flow through each of the inlet channel 106 and outlet channel 108 may be by capillary action. In some examples, it may be preferable to use e.g. a syringe or a pipette to help apply additional flow force to help dislodge any trapped embryos (or other developed material). This flow process is continued until all of the embryo(s) (or other developed material) have entered the inlet well 102 and can thus be retrieved from the device 100 via the open top 103 of the inlet well 102.

FIG. 2 illustrates another example of a portion of a microfluidic device 200. In this example, the device 200 is configured similarly to the device 100 of FIGS. 1a and 1b, but includes a plurality of outlet channels 208ₐ₋ₑ. In this example, there are five outlet channels 208, but other examples may include any number of outlet channels, for example, two, three, four, six, seven or eight outlet channels.

Each of the outlet channels 208 extend from the culture chamber 110 to the outlet well (not shown) similarly to the single outlet channel 108 described in relation to FIGS. 1a and 1b. Other features of the device 200 may be configured similarly to those described in relation to FIGS. 1a and 1b, so will not be described again in detail.

The plurality of outlet channels 208ₐ₋ₑ are each configured to prevent passage of a zygote stage embryo (or other cellular deposit) similarly to the outlet channel 108 in the example of FIGS. 1a and 1b. Each of the outlet channels 208ₐ₋ₑ aptly extend substantially in parallel to each other between the culture chamber 110 and the outlet well and may be substantially equally spaced apart. This can help to control fluid flow direction in the culture chamber and can help to disperse multiple embryos (or multiple cellular deposits) within the culture chamber 110. It will be appreciated that the plurality of outlet channels 208 converge to the single outlet well. In this way, each of the outlet channels converge to a single exit point (exit port).

In this example, each of the outlet channels 208ₐ₋ₑ have a substantially equal height sized to prevent the passage of a zygote stage embryo (or other cellular deposit). That is, the height of each outlet channel is less than the diameter of the zygote stage embryo (or other cellular deposit).

At least one of the plurality of outlet channels may have a different volume compared to another one of the plurality of outlet channels. For example, the height and/or width of each of the channels may be different to other channels to result in a different channel volume. In other examples, each of the plurality of outlet channels may have equal volume (i.e. each of the outlet channels may have the same volume).

For example, at least one of the plurality of outlet channels may have a different width compared to another one of the plurality of outlet channels. In other words, the width of some of the outlet channels 208ₐ₋ₑ may be different to the width of the other outlet channels. In this example, the two outermost channels 208ₐ, 208ₑ are of equal width and have the largest width of all the channels. The innermost central channel 208_{c} has the narrowest width and the channels either side of the innermost channel 208_{d}, 208_{b} are of equal width to each other and have a width between that of the outermost channels 208ₐ, 208ₑ and the innermost channel 208_{c}. In other words, the outlet channels increase in width from the innermost (central) channel to the outermost channels, thereby forming a symmetrical configuration of channels.

The total volume of the plurality of outlet channels is aptly equal to or substantially equal to the volume of the inlet channel.

The distribution and relative size (volume and/or width and/or height) of each of the outlet channels can help to determine the flow characteristics within the culture chamber 110. For example, the outlet channels can be configured to help distribute flow evenly throughout the culture chamber 110 and thereby encourage even distribution and separation of embryos within the culture chamber. Larger width outermost outlet channels compared to the innermost channel or channels can help to encourage flow toward the outer walls of the culture chamber, thereby reducing accumulation of embryos (or other cellular deposits) in the central region.

In other examples, a single outlet channel having a width equal to that of the culture chamber (i.e. the chamber radius for a circular chamber) may provide the optimum flow profile within the device. However, a plurality of channels may aptly be used to help prevent collapse of the device structure. This may particularly beneficial for device fabrication from some materials (for example, PDMS (polydimethylsiloxane)).

Aptly, each of the outlet channels may have a height:width ratio less than or equal to 1:10. This can help with both device fabrication and flow profile within the device.

In this particular non-limiting example, configured for use with mouse embryos (approximate diameter of the cellular deposit, which is an embryo at the zygote stage is 60 μm), the dimensions of the inlet channel, outlet channels, culture chamber and inlet and outlet wells are indicated in the table below.

| | Width | Height | Length | Volume | |
|---|---|---|---|---|---|
| Inlet channel | 250 μm | 200 μm | 7500 μm | 375 | nL |
| Outlet channels (a&e) | 200 μm | 30 μm | 7500 μm | 45 | nL |
| Outlet channels (b&d) | 150 μm | 30 μm | 7500 μm | 33.7 | nL |
| Outlet channel (c) | 100 μm | 30 μm | 7500 μm | 22.5 | nL |

| | Radius | Height | Volume |
|---|---|---|---|
| Culture chamber | 750 μm | 230 μm | 406 nL |
| Inlet/Outlet well | 750 μm | 5000 μm | 8.8 μL |

Aptly, in this example and other examples, each of the outlet channels is configured to have a height less than or equal to 50% of the diameter of the embryo at the zygote stage (or other cellular deposit) to prevent the embryo (or other cellular deposit) from escaping the culture chamber.

FIGS. 3a to 3d illustrate the fabrication of the microfluidic device of FIG. 2. Though the fabrication process described below may also be applicable to any other examples described herein.

As illustrated in FIG. 3a, the device 300 includes an upper layer 302 and a lower layer 304. Each of the upper layer 302 and the lower layer 304 may be formed from PDMS using a standard soft lithography technique, for example as described by D. Qin et al “Soft lithography for micro and nanoscale patterning,” Nat. Protoc., vol. 5, no. 3, pp. 491-502, March 2010. In other examples, the upper layers may be formed from any suitable material using soft lithography or other suitable techniques.

The upper layer 302 includes first and second apertures 306, 308 which correspond to the inlet well and outlet well of the device. On an underside of the upper layer 302, a channel 308 (or groove) is formed extending from the first aperture 306 towards the second aperture 308. The length of the channel 308 corresponds to the length of the inlet channel 106 of the device.

The underside of the upper layer also includes a recessed area 310, sized and shaped to correspond to an upper portion of the culture chamber 110 of the device. The recessed area 310 is continuous with the channel 308 such that the inlet channel of the device is fluidly coupled to the upper portion of the culture chamber.

An upper side of the lower layer 304 includes a first recess 312 corresponding to a lower portion of the culture chamber 110 and a second recess 314 corresponding to a lower portion of the outlet well. The upper side of the lower layer 304 also includes at least one groove extending between the first recess 312 and the second recess 314. Each of the at least one groove corresponds to a respective outlet channel of the device.

The upper layer 302 and the lower layer 304 are assembled in a face to face configuration to form the device as shown in FIGS. 3a and 3b. The assembly of the layers can also be seen in FIGS. 1a and 1b.

As shown in FIG. 3b a pipette 316 may be used to deposit a liquid (optionally containing a cellular deposit, e.g. one or more embryos) into the inlet well.

Although in the example shown in FIGS. 3a to 3d the device has a substantially rectangular outer shape, other outer shapes may also be suitable. For example, the device may have a substantially circular, oval, square, rectangular or other polygonal outer shape.

FIGS. 4a to 4c illustrate another example of a microfluidic device 400. The microfluidic device 400 is similar to the other devices described herein but additionally includes a reservoir 402. The reservoir 402 is defined between an outer wall 404 and an inner wall 406 spaced apart from the outer wall 404. The reservoir 402 is configured to extend around a central portion of the device including the inlet well, outlet well, culture chamber, inlet channel and at least one outlet channel. As such, the reservoir 402 encircles the central portion of the device. The reservoir 402 is configured to retain a volume of fluid therein. Filling reservoir 402 with fluid can help to avoid or reduce evaporation during development of the cellular deposit.

FIG. 4b is a side view of the device of FIG. 4a and illustrates an alternative configuration of the inlet well 103. In this example, the inlet well includes an inclined lower portion 103′. The inclined lower portion 103′ is inclined towards the opening of the inlet channel 106. The angle of inclination of the inclined portion with respect to the inlet channel may be between 0 and 90 degrees. For example, the angle of inclination may be about 45 degrees. The inclined portion 103′ may help to position a pipette in the inlet well and direct fluid from the pipette to the region directly adjacent the opening of the inlet channel 106.

In other examples, the outlet well and/or the inlet well may include a similarly inclined lower portion.

Any of the devices described herein may be used for culturing (supporting development of) at least one embryo. The at least one embryo may be a mammalian embryo. In some examples, the device may be used for simultaneously culturing a plurality of embryos, for example from 2 to 10 embryos, or from 4 to 6 embryos. The at least one embryo may be selected from a human or other primate, rodent, cattle, sheep or pig embryo.

A non-limiting example of a method of use of the device of any of FIGS. 1 to 4 (or any other examples described herein), for supporting development of a mouse embryo is as follows. The device is placed inside a 60 mm ART culture dish and surrounded with 4 mL embryo-tested water in the reservoir. This humidified environment obviates the need for large media reservoirs. Devices are prepared by adding a 10 μL KSOM drop to the inlet well and drawing the media through from the outlet well 10 times. 10 μL drops of fresh KSOM are then added to inlet and outlet before pre-equilibration overnight at 37° C. under 5% CO₂, 5% O₂ in humidified nitrogen. To load embryos, the 10 μl media drops are removed from the inlet well and outlet well before placing the embryos inside the inlet well at the inlet channel mouth using a 135 μm pipette tip. Media is then drawn through from the channel outlet until all embryos enter the central chamber by capillarity. 10 μL drops of pre-equilibrated KSOM are then added to inlet well and outlet well before culture at 37° C. under 5% CO₂, 5% O₂ in humidified nitrogen in a MINC benchtop incubator. At the end of the culture, embryos are retrieved by aspirating from the inlet well using the same 135 μm pipette tip until all the loaded embryos are retrieved. It will be appreciated that various aspects of the method may be adapted for supporting development of other cell deposits (including different species of embryo). For example, instead of KSOM, an alternative media may be used. Also, the culture environment may be adapted (e.g. different temperature, alternative gas ratios and/or combinations, alternative incubators etc.).

Various modifications to the detailed arrangements as described above are possible.

Although the flow of fluid through the device has been generally described above as flow by capillary action, in some examples flow of fluid (optionally containing embryos or other cellular deposit) may be initiated or aided by additional force, for example injecting manually using e.g. a syringe, or a pipette or providing extra fluid pressure at the inlet well and/or the outlet well. In other examples, a pump may be used to help provide a constant flow of fluid within the device.

Although the examples have been described above as being formed from PDMS, other materials may also be suitable, for example other plastics or polymers. For example, the device may be formed from a polystyrene. This may be particularly advantageous for an embodiment having a single outlet channel to help provide additional strength.

Although the culture chambers and the inlet and outlet wells described above are generally circular or oval in shape, different shaped chambers and wells may also be suitable. For example, the culture chamber and/or the inlet well and/or the outlet well may be substantially square, rectangular, elliptical, hexagonal, or any other regular or irregular polygonal shape.

Although the inlet channel has been described above as sized to allow passage of a blastocyst embryo, in other devices the inlet channel may be suitably sized to allow passage of the embryo (or other cellular deposit) at the stage at which is to be retrieved (the developed material). In general the cross-section of the inlet channel may be larger than a largest cross-section of the developed material be retrieved from the culture chamber.

In examples including a plurality of outlet channels, the width and/or height of the outlet channels may be configured in any suitable way. Aptly the height of the outlet channels may all be equal and the widths of each of the outlet channels may be selected according to desired flow characteristics within the culture chamber.

Although the height:width ratio of each of the outlet channels has been has been described above as less than or equal to 1:10, other ratios may be possible. For example, the height:width ratio may be from 1:20 to 1:5 or aptly from 1:15 to 1:8 or more aptly from 1:12 to 1:8.

Although the device is described herein as generally suitable for supporting development and/or culture of one or more embryos, the device may also be suitable for supporting development and/or culture of other, similar sized tissue types. For example, the device may be suitable for supporting development of or for culture of an oocyte (egg), ovarian follicles (such as primordial follicles or preantral follicles), cumulus oocyte complexes and ovarian tissue.

In some examples, such as the example illustrated in FIGS. 10a-c, the height of the culture chamber 510 is aptly equal to the height of the inlet channel 506. That is, the inlet channel 506 and the culture chamber 510 are coplanar (i.e. the upper and lower walls of the culture chamber 510 is coplanar with the upper and lower walls of the inlet channel 506, respectively). In other words, the inlet channel 506 extends from a lower portion of the inlet well 502 to the culture chamber 510, without a ‘step’ between the lower wall of the inlet channel 506 and the lower wall of the culture chamber 510. Such an arrangement may be beneficial in that, when manufacturing the device from two layers (in the manner described for FIG. 3) all the flow channel features can be produced within a single layer. In particular, the apertures and channels corresponding to the inlet well(s), inlet channel(s), culture chamber(s), outlet channel(s) and outlet well can be produced within the top layer 522, for example from a 3D printed mould. The bottom layer 524 can be left flat (i.e. without features thereon). In this manner, no alignment is needed during the assembly of the final device such that fabrication and assembly is simplified. In addition, the lack of a ‘step’ between the inlet channel and the culture chamber allows the embryos to be cultured to a larger size prior to extraction.

In some examples, such as that illustrated in FIGS. 11a and 11b, the device may include an outlet chamber fluidly coupling the culture chamber to the outlet well. The inclusion of an outlet chamber is particularly useful when the device includes more than one culture chamber (and similarly more than one inlet well and inlet channel). That is, the outlet chamber may be used to fluidly couple an outlet well with a number of culture chambers, each culture chamber being fluidly coupled to an inlet well. In such an arrangement, embryos can be individually cultured within separate culture chambers, but remain in communication to take advantage of the potential benefit of paracrine/autocrine factors.

In this example, the outlet chamber 650 of the device 600 fluidly couples culture chambers (in this example four culture chambers 610₁₋₄) to the outlet well via the outlet channel 608 (which in some examples may be multiple outlet channels). Each culture chamber 610₁₋₄ is fluidly coupled to a corresponding inlet well 603₁₋₄ via a corresponding inlet channel 606₁₋₄. It would be understood that the outlet chamber 650 may be used to fluidly couple any number of culture chambers to the outlet well 605. For example, the outlet chamber 650 may fluidly couple 2, 3, 4, 5 (as shown in FIG. 11c) or more culture chambers 610 to the outlet well 605.

In this example, the outlet chamber 650 extends from, or at least partially surrounds, a lower portion of each culture chamber 610₁₋₄. Aptly, a lower wall of each culture chamber 610₁₋₄ is coplanar with a lower wall of the outlet chamber 650. The outlet channel 608 extends from outlet chamber 650 towards the outlet well.

In this example the outlet chamber 650 is sized to prevent passage of the cellular deposit, aptly a zygote stage embryo, therethrough. That is, the outlet chamber 650 blocks the passage of the embryo 150 (i.e. the cellular deposit) therethrough, thereby acting to retain the embryo in the corresponding culture chamber 610₁₋₄. In this example, the height (i.e. thickness) of the outlet chamber 650 is less than a diameter of the zygote stage embryo.

In some examples the outlet channel 608 may also be sized to prevent passage of the cellular deposit therethrough. For example, the outlet channel 608 (or at least one of a plurality of outlet channels) may be the same height (i.e. thickness) as the outlet chamber 650. In other examples, outlet channel may have a greater height as the outlet chamber 650.

In some examples, the outlet chamber may be part of (i.e. incorporated into) the outlet channel. For example, the outlet chamber may be a flared or enlarged end of the outlet channel.

Alternative Devices

A device is described in detail herein that is suitable for supporting embryo development. However, similar devices may also be used to support the development of any cellular deposit (e.g. any cells and/or tissues, for example, cells and/or tissues that are derived from an ovary).

As used herein, the term “derived from an ovary” encompasses any cell and/or tissue that directly or indirectly originates from the ovary, including differentiated products thereof. For example, oocytes, ovarian follicles (such as primordial follicles, or cumulus-oocyte complexes, preantral follicles or antral follicles) and ovarian tissue (e.g. ovarian tissue fragments) are all encompassed by this term. In addition, embryos are encompassed, as they are originally derived from an ovarian product (the oocyte) and thus are indirectly derived from the ovary. As used herein, the term “embryo” encompasses zygote, zona pellucida, cleavage stage embryo, morula, and blastocyst.

As used herein, the term “supporting the development” refers to maintaining the viability of the cell and/or tissue that is deposited in the device and supporting cell division and/or cell differentiation of the cellular material. Cells and/or tissue that are deposited in the device are also referred to herein as the “cellular deposit” (i.e. the cellular material that is deposited into the device, prior to any cell division and/or cell differentiation within the device). Typically, the cellular deposit is a multicellular deposit (e.g. a multi-cellular product derived from the ovary).

Accordingly, the device is suitable for supporting the development of a cellular deposit (e.g. zygote stage embryo) comprising at least one of a cell or a tissue derived from an ovary.

Typically, the cellular deposit develops within the device (by cell growth, cell division and/or cell differentiation) to form a developed material. As used herein, the term “developed material” refers to the cells/and or tissue that are to be retrieved from the device.

Accordingly, there is provided a device for supporting the development of a cellular deposit comprising at least one of a cell or a tissue derived from an ovary, the device comprising:
an inlet well, an outlet well, and an enclosed culture chamber disposed between the inlet well and the outlet well;
an inlet channel fluidly coupling the inlet well to the culture chamber;
at least one outlet channel fluidly coupling the culture chamber to the outlet well;
wherein each of the at least one outlet channel is sized to prevent passage of the cellular deposit therethrough.

Suitably, the device may be a microfluidic device.

It will be appreciated that when supporting development of a cell or tissue derived from the ovary the outlet channel is sized to prevent passage of the cell or tissue initially deposited into the device (i.e. the cellular deposit) therethrough. As such, the outlet channel is sized to prevent the cellular deposit exiting the culture chamber.

As described in detail herein, when the cellular deposit is a zygote embryo, the outlet channel is sized to prevent passage of the zygote embryo therethrough. The zygote embryo is prevented from exiting the culture chamber, and may be retained within the device for a time sufficient to support development of the embryo (e.g. support cell division and/or differentiation of the zygote embryo into a further developed embryo such as a zona pellucida, morula or a blastocyst).

Similarly, when the cellular deposit is an oocyte, the outlet channel is sized to prevent passage of the oocyte therethrough. The oocyte is prevented from exiting the culture chamber, and may be retained within the device for a time sufficient to support development of the oocyte (e.g. support cell growth, cell division and/or differentiation of the oocyte into a fertile ovum).

Similarly, when the cellular deposit is an ovarian follicle, the outlet channel is sized to prevent passage of the ovarian follicle therethrough. The ovarian follicle is prevented from exiting the culture chamber, and may be retained within the device for a time sufficient to support development of the ovarian follicle (e.g. support cell growth, cell division and/or differentiation of the ovarian follicle).

Similarly, when the cellular deposit is an ovarian tissue, the outlet channel is sized to prevent passage of the ovarian tissue therethrough. The ovarian tissue is prevented from exiting the culture chamber, and may be retained within the device for a time sufficient to support development of the ovarian tissue (e.g. support cell growth, cell division and/or differentiation of the ovarian tissue).

Similarly, when the cellular deposit includes a zona pellucida, the outlet channel is sized to prevent passage of the cellular deposit (including zona pellucida) therethrough. The cellular deposit including the zona pellucida is prevented from exiting the culture chamber, and may be retained within the device for a time sufficient to support development of the cellular deposit including the zona pellucida (e.g. support cell division and/or differentiation of the cellular deposit/zona pellucida into a further developed embryo such as a morula or a blastocyst).

Similarly, when the cellular deposit is a morula, the outlet channel is sized to prevent passage of the morula therethrough. The morula is prevented from exiting the culture chamber, and may be retained within the device for a time sufficient to support development of the morula (e.g. support cell division and/or differentiation of the morula into a further developed embryo such as a blastocyst).

Similarly, when the cellular deposit is a blastocyst, the outlet channel is sized to prevent passage of the blastocyst therethrough. The blastocyst is prevented from exiting the culture chamber, and may be retained within the device for a time sufficient to support development of the blastocyst (e.g. support cell division and/or differentiation of the blastocyst into a further developed embryo such as a pre-implantation embryo).

Other features of the device described herein, for example the inlet well, the inlet channel and the culture chamber, may be similarly be sized or adapted according to the cellular deposit and/or the developed material. For example, the inlet well, inlet channel and the culture chamber may be sized to accommodate and/or allow passage of the developed material such that the developed material can be retrieved from the device.

Similar to the variation in size of the outlet channel discussed above, the skilled person will appreciate how the inlet well, inlet channel and culture chamber should be scaled to accommodate cellular deposits and/or developed material of different sizes. A non-limiting example of the dimensional considerations in relation to a mouse zygote (“cellular deposit”) and a mouse blastocyst (“developed material”) is described in detail herein and can be extrapolated for other cellular deposits (e.g. other mammalian zygotes) and developed materials (e.g. other mammalian blastocytes).

Other aspects of the invention are demonstrated by the following non-limiting examples.

### EXAMPLES

Materials and Methods

Microfluidic Device Design and Fabrication

Microfluidic devices were fabricated in polydimethylsiloxane (PDMS, Sylgard® 184, Down Corning, Mich., USA) using standard soft lithography technique. The procedure is carried in a Class 100 clean room with SU-8 2050 and 2035 negative photoresists. Briefly, 4″ silicon wafers (Si-Mat, Kaufering, Germany) were spin coated with SU-8 (target thicknesses 100, 200 and 30 μm). The lower the spin speed the harder is to get a flat layer, so the 200 μm layer was obtained by spin coating two layer of 100 μm instead of one unique layer. After baking, the resist layer was exposed to UV light through a 40,000 dpi printed Mylar mask (JD Photo Data, UK) and finally processed with SU-8 developer to remove unexposed resist after the wafer had been baked. Subsequently a 10:1 PDMS pre-polymer:curing agent mixture was prepared, poured on the SU8 mold, thoroughly degassed and left for curing overnight in oven at 70° C. Following this, the PDMS layer was cut using a sharp knife and released from the mold. Port to access the microfluidic chamber were opened by punching 1/16″ holes in the layer of PDMS with a stainless-steel round punch. Finally, the devices were assembled by bonding the 2 layers using oxygen plasma treatment (600 mT, 100 W, 40 s). This process allows for contamination removal (chemical), oxidation and activation of the surfaces, but acts also as antibacterial and antimicrobial treatment. Hence, once assembled, the devices were immediately filled with embryo tested water using disposable, sterile syringe and stored at 4° C. until used to preserve high level of humidity.

Computational Model

To evaluate and compare flow rate, velocity field and predict shear stress as function of microfluidic device geometry, fluid flow within it was simulated with COMSOL Multiphysics 5.2a.

To generate 3D model, the microfluidic design was first created by using computer-aided design (Autodesk AutoCAD 2017) software. The design geometry was then imported into COMSOL library. The fluid inside the device was simulated as an incompressible, homogeneous, Newtonian fluid with density (ρ=1000 kg m-3) and viscosity (μ=1×10-3 Pa s), as in previous studies. The COMSOL simulation also assumed that all flows in the described devices at the analysed flow rate are completely laminar, with a Reynolds number (Re) several orders of magnitude lower that the turbulent threshold (2300). The Reynolds number is conventionally defined as the ratio of the inertial forces to viscous forces. General assumption in microfluidics is a Reynolds number ≤1, due to the slow volumetric flow rate and the negligible inertial effects. The model was based on the steady-state Navier-Stokes equation for an incompressible Newtonian fluid:

ρ(v·∇)v=∇−p+η∇²v

where v and p are the velocity vector and the pressure. No slip boundary conditions were applied for the microdevice walls. A flat velocity profile was applied to the inlet channel, while a zero-pressure condition was applied to the outlet. Surface plot of velocity field in colour spectrum and line graph of velocity field fluctuation within the culture chamber were analysed. Shear rate (γ) at the bottom or top wall was also calculated by COMSOL, based on the velocity gradient right above the bottom or top wall. Share rate was then multiplied with the viscosity (μ) to determine the wall shear stress field (τ=μγ) within our microfluidic devices.

Flow Characterization

Fluorescent Beads Assay

To characterize the fluid flow within the device and trace the fluid dynamic changes in function of the different microdevice geometries of inlet and outlet channels (FIGS. 3a to 3d) 4.8 μm fluorescent polystyrene beads were used. The beads solution was perfused through the device thanks to capillarity phenomena using a Gilson pipette. In order create a flow toward the outlet, and simulate the experimental conditions when embryos/beads are manually loaded within the device, a drop of 3 ml was released in the inlet reservoir after emptying both inlet and outlet reservoirs. The flow was monitored and recorded by fluorescent microscope using a camera and software. Images and videos were taken in the 494/512 nm excitation/emission spectrum. Image frames were assessed measuring the length of the beads path divided by the exposure time using ImageJ software. The resulting velocities were used in COMSOL for shear stress evaluation of the fluids within the microdevices.

Fluorescein

For further evaluation of flow within the culture chamber in function of the inlet and outlet channels a solution of fluorescein 0.05 mg mL⁻¹ was used. A microdrop of 3 mL was released in the inlet reservoir as previous explained and a fluorescence microscope with a camera and software was used for time lapse evaluation of the fluorescein flow chamber profile over time.

Microfluidic Device Loading and Retrieval Testing

75.4 μm polystyrene beads (PPX-800-10, 5% w/v) were manually loaded and retrieved using EZ-Grip pipettes (RI consumables) to simulate experimental mouse embryo behaviour under loading and retrieval flow effect respectively, and to test handling of the microfluidic devices. Beads spreading during loading and beads retrieval from the chamber were assessed for different designs of inlet and outlet channels with the support of a microscope and the acquisition software.

Murine Embryo Production

Murine embryos from the strain C57/B6 were produced at MRC Harwell according to established protocols. Mature murine M2 Oocyte-cumulus-complexes (OCCs) were retrieved following ovarian hyper-stimulation according to REF. In-Vitro Fertilisation (IVF) was performed. Presumptive zygotes were cryopreserved following IVF and transported to the University of Leeds by dry shipper.

Murine Embryo Culture and Retrieval

Murine presumptive zygotes were thawed following MRC protocols. Briefly, embryo straws were held in air for 30 s, then plunged into room temperature water until the contents had visibly thawed (around 10 s). The straws were cut at the seal and the plug bisected before pushing the contents into a 60 mm IVF hydrophobic culture dish with a straw. Embryos were incubated for 5 min before 2×5 min washes in 100 μL M2 medium at 37° C. Embryos were then washed through 3×10 μL drops of preequilibrated KSOM under oil before culture.

Double Channel Device

Double channel devices were equipped with reservoirs to the inlet and outlet (inlet and outlet wells). The inlet well was filled with 250 μL KSOM and media was drawn through from the outlet well 10 times. The media in both reservoirs was then replaced with 250 μL fresh media and preequilibrated overnight. At the time of embryo loading, 250 μL media was removed from each reservoir. Embryos were placed inside the inlet at the channel mouth using a 135 μL pipette tip. Media was then drawn through from the channel outlet until all embryos entered the central culture chamber. The reservoirs were then filled with 250 μL pre-equilibrated KSOM media.

At the time of embryo retrieval, 250 μL media was removed from the inlet reservoir and the embryos were drawn through and collected at the inlet well using a 135 μm pipette tip. The devices were then cultured at 37° C. under 5% CO_{2, 5}% O₂ in humidified nitrogen in a conventional incubator.

Wide Channel Device

The wide channel device was placed inside a 60 mm ART culture dish and surrounded with 4 ml embryo-tested water. This humidified environment obviated the need for large media reservoirs. Devices were prepared by adding a 10 μL KSOM drop to the channel inlet and drawing the media through from the channel outlet 10 times. 10 μL drops of fresh KSOM were then added to inlet and outlet before pre-equilibration overnight at 37° C. under 5% CO₂, 5% O₂ in humidified nitrogen. To load embryos, the 10 μl media drops were removed from the inlet and outlet reservoirs before placing the embryos inside the inlet at the channel mouth using a 135 μm pipette tip. Media was then drawn through from the channel outlet until all embryos entered the central chamber. 10 μL drops of pre-equilibrated KSOM were then added to channel inlet and outlet before culture at 37° C. under 5% CO₂, 5% O₂ in humidified nitrogen in a MINC benchtop incubator. Embryo loading was equally successful and practical using a range of bulb and pipettor embryo handling devices common to clinical and research ART laboratories.

Results and Discussion

Microfluidic Structure Design

In order to closely mimic physiological journey of an embryo within the oviduct from the zygote until the blastocyst stage, microfluidic devices were designed to include (i) a total volume (reservoir not included) less than 1 μL (5-50 μL is the volume currently used for culture medium drop in more traditional culture technique), (ii) one inlet and (iii) one outlet reservoir connected to a culture chamber via two microfluidic channels with different thickness positioned at two different levels, and (iv) a narrow outlet channel with a thickness of 30 μm, which is quite smaller than the size of mouse embryo (diameter ˜100 μm) necessary to prevent them from flowing out during the loading when a flow is applied. This microfluidic structure was obtained by bonding together two layer of PDMS (FIG. 3).

The dimensions of the designs were adapted to work with mouse embryos (diameter ˜100 μm at the blastocyst stage which may expand to more than 150 μm at the hatched blastocyst stage), and to minimize wall shear stress (WSS) within the channels during loading and retrieval (1.2 dyn cm⁻² shear stress caused lethality within 12 h for blastocysts). In order to optimize flow inside the microfluidic device, and facilitate embryo loading/retrieval, reducing the possibility of embryo/wall contact and so the stress on them, more designs that include two different inlet channel rectangular cross-section were evaluated (FIG. 5).

Flow in microfluidic devices can be described by the incompressible Navier-Stokes equation for uniform-viscous Newtonian fluids with no body forces, and is determined by the volumetric flow rate and the geometry of the microfluidic structure. In order to size a microfluidic device as function of the maximum tolerable shear rate, a relationship between volumetric flow rate (Q), geometric dimension and shear stress for a pipe with a circular cross-section or wide rectangular cross-section (height-width ratio ˜0) can be easily found as previously shown. Due to the microfabrication process, flow channels in microsystems are usually rectangular with the height (H) comparable to the width (VV). In this case, the shear rate can be calculated as the summation of a Fourier series. The resulting equation takes the following form:

$$τ = μ ( \frac{6 Q}{{WH}^{2}} ) ( 1 + \frac{H}{W} ) f ( \frac{H}{W} )$$

where the function f(x) is given as follows:

$$f ( x ) = {[ {( 1 + \frac{1}{x} )}^{2} ( 1 - \frac{192}{{π}^{5} x} {\sum }_{i = 1 , 3 , 5}^{∞} \frac{tanh ( \frac{π}{2} ix )}{{i}^{5}} ) ]}^{- 1}$$

According to the equations above, and considering the square sections of the inlet channels, the flow rates necessary to guarantee WSS below the harmful threshold value of 1.2 dyn cm⁻² were calculated for both inlet channel designs (reported in FIG. 5).

The estimated flow rate values agreed with the typical flow rates used in microfluidic networks, where laminar flow and absence of turbulence are essential to minimize unsteady-state flows at bending and branching points. As expected, at the same flow rate, the Design 2 allowed to obtain WSS in the inlet channel about 13-fold smaller compared to Design 1 (0.087 dyn cm⁻² and 1.2 dyn cm⁻² respectively).

Bigger channel cross-section and lower hydraulic resistance (Rₕ) result in lower velocity within the culture chamber and reduce the shear stress in the inlet channel below the lethal WSS values.

Computational Model on COMSOL Multiphysics 5.2a.

To evaluate how the outlet channels geometry can affect flow rate, and velocity field within the whole device, a flow rate of 1.014 μL min⁻¹ was applied on COMSOL simulations of the proposed design (Design 2.1-Design 2.4).

As shown in FIG. 6, during loading, the velocity profile at the inlet channel (hydraulic resistance ∝10¹⁰ Pa s m⁻¹) is first flattened down by the presence of the culture chamber and then risen again above the initial value in the outlet channel, due to increased hydraulic resistance (up to 10¹²⁻¹³ Pa s m⁻¹). The difference in colour tone from warm to cold are representative of the decreasing hydraulic resistance within the outlet channels from Design 2.1 to Design 2.4 (FIG. 6).

Between the proposed designs, Design 2.4 imposes the lower hydraulic resistance to the flow and, being the outlet channels disposed around half of the chamber, it shows the wider velocity profile within the culture chamber along the cross-sectional direction (FIG. 8).

According with the computational results found on COMSOL, and following the standard process of photolithography, the device with Design 1 and Design 2.4 were fabricated and assembled for further characterization.

Flow Characterization and Shear Stress Analysis

Flow within the devices was characterized for the Design 1 and Design 2.4 as a function of the inlet and outlet channels dimensions with the help of 4.8 μm fluorescent polystyrene beads. Determination of fluid velocity and trace of fluidic dynamics of the microdevice is essential to characterize culture conditions in the proposed design. Flow was created by manual loading using a flexi pipette (EZ-Grip). The maximal velocity values for the inlet channel for Design 1 and 2.4 were estimated to be respectively 0.25 mm s⁻¹ and 0.4 mm s⁻¹, which correspond to a flow rate of 0.156 μL min⁻¹ (Re=0.026) and 1.17 μL min⁻¹ (Re=0.087). As expected, due to lower hydraulic resistance, Design 2.4 was characterized by a higher flow profile compared to Design 1, which can be helpful during embryo loading and retrieval, and both devices showed a lower flow rate compared to the theoretical threshold values (1.014 μL min⁻¹ and 13.91 μL min⁻¹ respectively) necessary to have WSS lower than lethal value of 1.2 dyn cm⁻².

The estimated inlet velocities were applied to a COMSOL model to predict the shear stress in the microfluidic system (FIG. 9). Both Design 1 and Design 2.4 were characterized by shear rates well below the threshold value, with some critical point (˜3.5 dyn cm⁻²) close the outlet channels mouth where the WSS exceeds the harmful value. However, as demonstrated by biological test with embryos reported below, these critical values do not affect embryo viability due to the short handling time (few seconds) and to the point that these values are reached in sections where the embryos cannot arrive (channel thickness 30 μm<average embryo diameter).

Once the velocity has been assessed, to better characterize flow profile inside the microfluidic chambers a solution of fluorescein was flowed in the chamber (FIG. 7). As predicted from the computational analysis, the microfluidic device with Design 2.4 gives a wider and higher profile (FIG. 7). Considering the better diffusion of nutrient factors within the chamber (e.g. fluorescein), and the transient mechanical stimulation that is exerted on the embryos, these results suggest that the embryos homogeneously spread in the chamber and receive equal amount of nutrient. Additionally, if the medium has to be refreshed during the embryo development, the full volume of the chamber is refreshed in Design 2.4.

75.4 μm polystyrene beads were instead used to simulate embryos behaviour during loading and retrieval test. During manual loading beads were not pushed through the inlet channel, but gently released at the mouth of it to be then transported inside the culture chamber from the flow by capillarity (physical phenomenon enhanced from higher flow rate). The greater flow rate (1.17 μl min⁻¹) within the devices with design 2.4 in fact, made them much easier to be used. As expected beads within the microfluidic chamber of the Design 2.4 tended also to be better spread throughout the whole width of the chamber, compared to Design 1 (FIG. 7). This allowed to avoid contact between embryos and to maintain instead a reciprocal distance that favours paracrine signalling between embryos, and avoid detrimental accumulation of secreted products in the medium surrounding them.

Evaporation and Osmolarity Changes in Device Culture

Due to evaporative losses in preliminary tests, media reservoirs holding 250 μL KSOM were used with the Design 2 devices. Devices were cultured in large Petri dishes with daily media replenishment, surrounded with embryo-tested water, or without humidification or media replacement. The reservoir volume did not change in reservoirs coated with oil (250 μL±0), however volume significantly decreased in all other conditions. Devices with daily (202 μL±6) or 2-day (193 μL±3.3) media replacement had significantly lower volume than devices surrounded with water (220 μL±5, p<0.05). However, osmolality was not significantly increased in water-flooded devices compared to oil-coated reservoirs. Osmolality was increased after 3 days of culture in devices with 2-day (336 mOsm kg⁻¹±10) or daily media replacement (298 mOsm kg⁻¹±12) compared to oil-coating (252 mOsm kg⁻¹±6.4), and was increased in 2-day but not daily media replacement compared to water-flooded devices (261 mOsm kg⁻¹±1.2). In both media replenishment and humidified petri dishes, evaporative losses and increases in osmolarity were significantly smaller compared to the control devices.

Embryo Development in Double Channel Device

Embryos were cultured in Design 2 devices with daily media replacement of the 250 μL reservoir or surrounded with water in large Petri dishes to reduce evaporation. There was no difference in cleavage or blastocyst rate between these device conditions. Device cleavage rate (94.44±2.53, n=15) and blastocyst rate (90%±6.3) were not statistically different to microdrop culture (94.73%±1.563, n=13 and 94%±1.8, p=0.3684 respectively).

Embryo Development in Wide Channel Device

The wide channel devices (Design 2.4) were placed inside 60 mm culture dishes and surrounded with embryo tested water. The resulting humidified microenvironment obviated the need for large media reservoirs and 10 μL KSOM drops were instead added to the channel inlet and outlet during culture. The total volume of these drops did not change significantly during a 3-day culture period, although media tended to flow to the outlet side by passive pumping.

Embryo development was not significantly different to drop culture, with similar cleavage rate (93.94%±3.2, n=11 vs control 94.73%±1.6, n=11, p=0.8), blastocyst rate (96%±2.1 vs control 94%±1.8, p=0.37) and hatching rate (11.11%±3.5, n=4 vs control 20.83%±9.5, n=5, p=0.33).

Conclusions

In this study, the inventors have proposed a closed novel and optimized microfluidic system able to support in vitro embryo growth and to improve the developmental competence of the in vitro manipulated embryo. Inlet channel-culture chamber-outlet channel structured devices were designed, and different cross-section for both inlet and outlet channels studied and compared in term of flow rate, velocity field, shear stress, for their performance on embryo loading, growing and retrieval. Theoretical methods and computational simulations were firstly carried out and subsequently confirmed by practical experiments with beads and embryos. What the inventors found was that a wider inlet channel ensures a lower velocity and WSS profile as well as a wider flow profile within the microculture chamber when the same flow rate is applied. This allowed a wider embryo spreading during loading and a more efficient embryo recovery during retrieval. Embryos can be also easily retrieved from the microfluidic chamber of the present invention, by emptying the inlet reservoir to create a back-flow in the chamber, to be then subjected to further evaluations.

Incubating the devices inside 60 mm IVF Petri dishes, flooded with embryo tested water, prevented significant evaporative losses. Embryo toxicity was tested by the industry standard mouse embryo assay. Embryo development was similar to control microdrops, with 94.4% vs 94.7% cleavage and 96% vs 94% blastocyst rate. This data indicates that the present devices are entirely embryo-safe, supporting successful embryo development to the blastocyst stage without the need for media changes or oil overlay.

In human IVF, usually embryos are developed in vitro to be later implanted in the uterus. Unfortunately, IVF is currently associated with two important issues that derive, at least in part, from the inability to adequately support the in vitro development of the embryo through the different stages and to assess the reproductive potential on individual embryos: (i) low implantation rate, and (ii) high multiple pregnancy rate. The present microfluidic device also aims to address these problems, by improving manipulated embryo quality to reduce use of animals, and favour the use of Non-Surgical Embryo Transfer techniques which can only be used to transfer late stage embryos.

Other aspects are described in the following numbered paragraphs.

1. A microfluidic device for supporting embryo development, the device comprising:
an inlet well, an outlet well, and an enclosed culture chamber disposed between the inlet well and the outlet well;
an inlet channel fluidly coupling the inlet well to the culture chamber;
at least one outlet channel fluidly coupling the culture chamber to the outlet well;
wherein each of the at least one outlet channel is sized to prevent passage of a zygote stage embryo therethrough.

2. A microfluidic device according to paragraph 1, wherein the inlet channel is configured for flow of fluid by capillary action between the inlet well and the culture chamber.

3. A microfluidic device according to paragraph 1 or paragraph 2, wherein each of the at least one outlet channel is configured for flow of fluid by capillary action between the culture chamber and the outlet well.

4. A microfluidic device according to any preceding paragraph, wherein the inlet channel extends from a lower portion of the inlet well to an upper portion of the culture chamber.

5. A microfluidic device according to any preceding paragraph, wherein a lower wall of the inlet well is coplanar with a lower wall of the inlet channel.

6. A microfluidic device according to any preceding paragraph, wherein an upper wall of the inlet channel is coplanar with an upper wall of the culture chamber.

7. A microfluidic device according to any preceding paragraph, wherein each of the at least one outlet channel extends from a lower portion of the culture chamber to a lower portion of the outlet well.

8. A microfluidic device according to any preceding paragraph, wherein a lower wall of the culture chamber is coplanar with a lower wall of each of the at least one outlet channel.

9. A microfluidic device according to any preceding paragraph, wherein the lower wall of each of the at least one outlet channel is coplanar with a lower wall of the outlet well.

10. A microfluidic device according to any preceding paragraph, wherein a height and/or a width of each of the at least one outlet channel is less than a diameter of the zygote stage embryo.

11. A microfluidic device according to paragraph 10, wherein the height and/or the width of each of the at least one outlet channel is less than or equal to 50% of the diameter of the zygote stage embryo.

12. A microfluidic device according to any preceding paragraph wherein the inlet channel is sized to allow passage of a blastocyst stage embryo.

13. A microfluidic device according to paragraph 12, wherein a smallest cross-sectional dimension of the inlet channel is at least 1.5 times larger than the diameter of a blastocyst stage embryo.

14. A microfluidic device according to any preceding paragraph, wherein the culture chamber is substantially circular, oval or elliptical in shape and has a constant height.

15. A microfluidic device according to any preceding paragraph, wherein a diameter or width of the culture chamber is greater than or equal to a width of the inlet channel.

16. A microfluidic device according to any preceding paragraph, wherein the culture chamber has a volume from about 3 nL to about 100 μL.

17. A microfluidic device according to any preceding paragraph, wherein the inlet well and the outlet well each have a volume from about 2 to about 20 μL.

18.A microfluidic device according to any preceding paragraph, wherein the inlet well comprises an inclined lower portion, wherein the inclined lower portion is inclined towards the opening of the inlet channel at an angle between 0 and 90 degrees.

19. A microfluidic device according to any preceding paragraph, wherein the inlet channel has a volume substantially equal to a total volume of the at least one outlet channel.

20. A microfluidic device according to any preceding paragraph, comprising a plurality of outlet channels.

21. A microfluidic device according to paragraph 20, wherein the plurality of outlet channels each extend substantially in parallel between the culture chamber and the outlet well.

22. A microfluidic device according to paragraph 20 or paragraph 21, wherein at least one of the plurality of outlet channels has a different volume compared to another one of the plurality of outlet channels.

23. A microfluidic device according to any preceding paragraph, further comprising a reservoir defined between an outer wall of the device and an inner wall spaced apart from the outer wall, wherein the reservoir extends around the inlet well, outlet well, culture chamber, inlet channel and at least one outlet channel and is configured to retain a volume of fluid therein.

24. Use of the device of any preceding paragraph for culturing at least one embryo.

25. Use of the device according to paragraph 24, wherein the at least one embryo is a mammalian embryo, optionally wherein the at least one embryo is selected from a human or other primate, rodent, cattle, sheep or pig embryo.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A device for supporting development of a cellular deposit comprising at least one of a cell or a tissue derived from an ovary, the device comprising:
an inlet well, an outlet well, and an enclosed culture chamber disposed between the inlet well and the outlet well;
an inlet channel fluidly coupling the inlet well to the culture chamber;
an outlet chamber and/or at least one outlet channel fluidly coupling the culture chamber to the outlet well;
wherein at least one of the outlet chamber and the at least one outlet channel is sized to prevent passage of the cellular deposit therethrough.

2. A device according to claim 1, wherein the inlet channel is configured for flow of fluid by capillary action between the inlet well and the culture chamber.

3. A device according to claim 1, wherein each of the at least one outlet channel is configured for flow of fluid by capillary action between the culture chamber and the outlet well.

4. A device according to claim 1, wherein the inlet channel extends from a lower portion of the inlet well to an upper portion of the culture chamber.

5. A device according to claim 1, wherein a lower wall of the inlet well is coplanar with a lower wall of the inlet channel

6. A device according to claim 1, wherein an upper wall of the inlet channel is coplanar with an upper wall of the culture chamber.

7. A device according to claim 1, wherein each of the at least one outlet channel extends from a lower portion of the culture chamber to a lower portion of the outlet well.

8. A device according to claim 1, wherein a lower wall of the culture chamber is coplanar with a lower wall of each of the at least one outlet channel

9. A device according to claim 1, wherein the lower wall of each of the at least one outlet channel is coplanar with a lower wall of the outlet well.

10. A device according to claim 1, wherein a height and/or a width of each of the at least one outlet channel is less than a diameter of the cellular deposit.

11. A device according to claim 10, wherein the height and/or the width of each of the at least one outlet channel is less than or equal to 50% of the diameter of the cellular deposit.

12. A device according to claim 1, wherein the inlet channel is sized to allow passage of a developed material.

13. A device according to claim 12, wherein a smallest cross-sectional dimension of the inlet channel is at least 1.5 times larger than the diameter of the developed material.

14. A device according to claim 1, wherein the culture chamber is substantially circular, oval or elliptical in shape and has a constant height.

15. A device according to claim 1, wherein a diameter or width of the culture chamber is greater than or equal to a width of the inlet channel.

16. A device according to claim 1, wherein the culture chamber has a volume from about 3 nL to about 100 μL.

17. A device according to claim 1, wherein the inlet well and the outlet well each have a volume from about 2 to about 20 μL.

18. A device according to claim 1, wherein the inlet well comprises an inclined lower portion, wherein the inclined lower portion is inclined towards the opening of the inlet channel at an angle between 0 and 90 degrees.

19. A device according to claim 1, wherein the inlet channel has a volume substantially equal to a total volume of the at least one outlet channel.

20. A device according to claim 1, comprising a plurality of outlet channels.

21. A device according to claim 20, wherein the plurality of outlet channels each extend substantially in parallel between the culture chamber and the outlet well.

22. A device according to claim 20, wherein at least one of the plurality of outlet channels has a different volume compared to another one of the plurality of outlet channels.

23. A device according to claim 20, wherein each of the plurality of outlet channels have equal volume.

24. A device according to claim 1, further comprising a reservoir defined between an outer wall of the device and an inner wall spaced apart from the outer wall, wherein the reservoir extends around the inlet well, outlet well, culture chamber, inlet channel and at least one outlet channel and is configured to retain a volume of fluid therein.

25. Use of the device of claim 1 for supporting development of a cellular deposit comprising at least one of a cell or a tissue derived from an ovary.

26. Use of the device according to claim 25, wherein the cell or the tissue derived from the ovary is selected from an oocyte, an ovarian follicle, an ovarian tissue and an embryo, optionally wherein the embryo is selected from a zygote, a cleavage stage embryo, a morula, a blastocyst.
